(19) United States
(12) Patent Application Publication
(10) Pub. No.: US 2023/0096303 A1
(43) Pub. Date: Mar. 30, 2023

(54) A Consumable Product Comprising Malted Cereals for Promoting Recovery at Physical Activity

(71) Applicant: LANTMÄNNEN FUNCTIONAL FOODS AB, Stockholm (SE)

(72) Inventor: Leif Göransson, Kågeröd (SE)

(73) Assignee: LANTMÄNNEN FUNCTIONAL FOODS AB, Stockholm (SE)

(21) Appl. No.: 17/910,175

(22) PCT Filed: Mar. 26, 2021
(86) PCT No.: PCT/EP2021/057975

§ 371 Date: Sep. 8, 2022

(30) Foreign Application Priority Data
Mar. 26, 2020 (SE) 2030098-4

(51) Int. Cl. A23L 7/20; A23L 33/105

(52) U.S. Cl. CPC A23L 7/20; A23L 33/105

(57) ABSTRACT

The present invention relates to a consumable product comprising malted oats and/or a leachate of malted oats for use, if consumed in an amount sufficient enough to induce endogenous production of Protein Antisecretory Factor (Protein AF) and/or fragments thereof in a mammal after consumption, in increasing cardiac output and/or decreasing the recovery process of a mammal during and/or after physical activity, thereby treating and/or preventing muscle damage caused by strains and/or tears in an athletic mammal, as well as for preventing paralysis during and/or after physical activity. Said consumption of said consumable product before during and/or after said physical activity has for the first time been found to stabilize the Erythrocyte Fluid Volume fraction (EFV) of a mammal during and/or after physical activity at a value of no more than 3% over resting value, such as at a value between 37-38%, and to stabilize the creatine kinase value of said mammal at a lower value than for mammals not consuming SPC, i.e. at values which indicate reduced muscle stress and damage.

The use of the consumable product is intended as food or feed for humans and/or animals, such as but not limited to horses and dogs.

### TECHNICAL FIELD

The present invention relates to a consumable product comprising malted oats and/or a leachate of malted oats for use, if consumed in an amount sufficient enough to induce endogenous production of Protein Antisecretory Factor (Protein AF) and/or fragments thereof in a mammal after consumption, in increasing cardiac output in said mammal, decreasing the time needed for muscle recovery of a mammal during and/or after physical activity, effectively treating and/or preventing muscle damage caused by strains and/or tears in an athletic mammal, as well as preventing paralysis during and/or after physical activity. Said consumption of said consumable product before during and/or after said physical activity has for the first time been found to stabilize the Erythrocyte Fluid Volume fraction (EFV) of a mammal during and/or after physical activity at a value of no more than 3% over resting value, such as at a value between 37-38%, and to stabilize the creatine kinase value of said mammal at a lower value than for mammals not consuming the consumable product, i.e. at values which indicate elevated oxygen uptake capacity in blood and reduced muscle stress and damage.

The use of the consumable product is intended as food or feed for humans and/or animals, such as but not limited to horses and dogs.

It is herein for the first time disclosed that a consumable product, comprising malted oats and/or a leachate of malted oats in an amount sufficient enough to induce endogenous production of Protein Antisecretory Factor (Protein AF) and/or fragments thereof in a mammal after consumption, stabilizes hemoglobin, hematocrit and/or creatine kinase responses in a mammal during and/or after physical activity, this in turn is indicative of an increased cardiac output.

The malted oats and/or a leachate of malted oats comprised in the consumable product for use according to the present invention comprise in particular avenanthramide D at a concentration which is at least 100% higher as compared to in the corresponding non-malted oats and is obtained from a malting process comprising the steps of wet steeping and germinating oat at a temperature from about 5° C. to about 20° C., and subsequent drying said oat at no more than 80° C. air temperature.

Although the present invention is based on the surprising findings of the effects of malted oats in particular, it stands to reason that other malted cereals with the ability to induce endogenous production of Protein Antisecretory Factor (Protein AF) and/or fragments thereof in a mammal after consumption will also be able to stabilize hemoglobin, creatine kinase as well as hematocrit responses in a mammal during and/or after physical activity.

### BACKGROUND

It is a long felt need to increase cardiac output, to prevent muscle damage during exercise and to speed up muscle recovery following exercise in humans as well as in animals, in particular in athletes and in athletic animals. Albeit, this is true not only for athletic populations but also for individuals who participate in strenuous exercises as part of their lifestyle or work.

Cardiac output (CO), also known as heart output is a term used in cardiac physiology that describes the volume of blood being pumped by the heart, by the left and right ventricle, per unit time. Cardiac output (CO) is the product of the heart rate (HR), i.e. the number of heartbeats per minute (bpm), and the stroke volume (SV), which is the volume of blood pumped from the ventricle per beat; Values for cardiac output are usually denoted as L/min. For a healthy person weighing 70 kg, the cardiac output at rest averages about 5 L/min; assuming a heart rate of 70 beats/min, the stroke volume would be approximately 70 ml.

Because cardiac output is related to the quantity of blood delivered to various parts of the body, it is an important component of how efficiently the heart can meet the body's demands for the maintenance of adequate tissue perfusion. Body tissues require continuous oxygen delivery which requires the sustained transport of oxygen to the tissues by the systemic circulation of oxygenated blood at an adequate pressure from the left ventricle of the heart via the aorta and arteries. Oxygen delivery (DO2 mL/min) is the resultant of blood flow (cardiac output CO) times the blood oxygen content (CaO2). The amount/percentage of the circulated oxygen consumed (VO2) per minute through metabolism varies depending on the activity level but at rest is circa 25% of the DO2. Physical exercise requires a higher than resting-level of oxygen consumption to support increased muscle activity. During exercise, the cardiac output increases more than the total resistance decreases, so the mean arterial pressure usually increases by a small amount. Pulse pressure, in contrast, markedly increases because of an increase in both stroke volume and the speed at which the stroke volume is ejected. Cardiac output increases significantly during maximal exercise effort due to the increase in stroke volume. Regular aerobic fitness, anaerobic fitness, and muscular endurance training over time results in increased cardiac output which in turn results in greater oxygen supply during exercise, waste removal and hence improved endurance performance.

During physical exercise, several factors influence muscle performance, preventing muscle damage and effecting recovery time after performance. In one aspect, ready availability of insulin is essential, which is also necessary in the post exercise recovery process in replenishing glycogen and in the rebuilding and repair of muscle protein. Protein and specific amino acids can stimulate the insulin response and thereby speed up muscle recovery. Free radicals also play an important role in exercise induced muscle damage, thus anti-oxidants can reduce muscle damage and help maintain cell integrity by reducing oxidative stress. Furthermore, amino acids and certain natural supplements can help minimize muscle stress during exercise.

Recovery from exercise requires restoration of fluid (hydration) and electrolytes, rapid replenishment of muscle glycogen, reduction of oxidative and muscle stress and rebuilding and repairing of muscle protein damaged incurred during exercise. In particular, the glycogen stores in the mammalian body need to be replenished and any muscle cells damaged during exercise to be repaired. To that end, research in humans and horses has shown that ingesting specific amino acids after exercise can decrease muscle recovery time.

In particular, all performance athletes, including performance horses and human athletes, must strive to maintain proper hydration to transport materials to and from the cells within the body and to synthesize and repair body tissues. The amount of water required depends on the amount of water lost from the body and the amount of water utilized for synthesis of protein. During physical activity, water is lost from the body primarily in sweat, urine, and feces. Thus, to shorten the recovery time after physical activity and/or to minimize dehydration during physical activity it would be desirable to be able to balance cellular transport of fluids across the cell barrier during and/or after the physical activity.

### Hemoglobin—Erythrocyte Fluid Volume (EFV)

What is more, during exercise, the cardiovascular system has to warrant substrate supply to working muscles. The main function of red blood cells in exercise is the transport of O₂ from the lungs to the tissues and the delivery of metabolically produced CO₂ to the lungs for expiration. Further, hemoglobin also contributes to the blood's buffering capacity, and ATP and NO release from red blood cells contributes to vasodilation and improved blood flow to working muscle. These functions require adequate amounts of red blood cells in circulation. Therefore, the increased demand for oxygen is met by increasing muscle blood flow and by improved O₂ unloading from Hemoglobin (Hb), achieved by decreasing Hb-O₂ affinity.

Parameters required to evaluate O₂ transport capacity are the Hb concentration in blood (cHb) and hematocrit (Hct), as well as total Hb mass (tHb) and total red blood cell volume (Erythrocyte Fluid Volume fraction/tEFV) in circulation. cHb and Hct are easy to measure with standard hematological laboratory equipment. Together with CO₂, they indicate the amount of O₂ that can be delivered to the periphery per unit volume of cardiac output.

Changes in Hct occur rapidly. Hct increases during exercise when fluid replacement during exercise is insufficient. There is fluid loss due to sweating, a shift of plasma water into the extracellular space due to the accumulation of osmotically active metabolites, and filtration as a consequence of an increased capillary hydrostatic pressure. The resultant increase in plasma protein increases oncotic pressure and thus moderates fluid escape.

The magnitude of Hct change is depend on exercise intensity during training sessions and the type of exercise (strength vs. endurance). Changes appear e.g. less pronounced during swimming than running exercise.

The increased tHb and tEFV in trained athletes indicates that exercise stimulates erythropoiesis. An additional marker is the elevation of reticulocytes counts which can be observed within 1-2 days after endurance and strength training units.

Many studies have shown that Hct tends to be lower in athletes than in sedentary individuals which has led to an established reference Hct and Hb values for athletes. Approx. 85% of the female and 22% of the male athletes have Hct values below 44%.

In conclusion, there are many mechanisms that contribute to an increased tissue oxygen supply during exercise. They involve adjustments during exercise and to training. Training increases total hemoglobin mass by stimulating erythropoiesis, which increases the amount of O₂ that can be carried by blood. It also increases red blood cell 2,3-DPG, which increases the sensitivity of Hb-O₂ affinity to acidification dependent O₂-release.

### Creatine Kinase

Physical exercise or strenuous sporting activities can further increase blood creatine kinase (CK) levels. CK levels respond to marked changes in the amount and intensity of exercise. Thus, CK levels may increase significantly after unusual and eccentric types of exercise. This primarily applies to strength and speed-strength exercise stress. The appearance of creatine kinase (CK) in blood is generally considered to be an indirect marker of muscle damage, particularly for diagnosis of medical conditions such as myocardial infarction, muscular dystrophy, and cerebral diseases.

Patients with suspected exercise-induced CK increases are often advised to observe a training break of one week. Unfortunately, competitive athletes often find it quite impossible to do this.

### The Antisecretory Factor (AF)

The antisecretory factor (AF) is a class of proteins that occurs naturally in the body. Protein Antisecretory Factor (Protein AF) is a 41 kDa protein that was originally described to provide protection against diarrhoea diseases and intestinal inflammation (for a review, see Lange and Lönnroth, 2001). The Protein Antisecretory Factor (Protein AF) has long since been sequenced and its cDNA cloned (see WO 97/08202). The antisecretory activity seems to be mainly exerted by a peptide located between the amino acid positions 35 and 50 on the Protein Antisecretory Factor (Protein AF) sequence which comprises at least 4-16, such as 4, 6, 7, 8 or 16 amino acids of the consensus sequence. The biological effect of AF is exerted by any peptide or polypeptide comprising at least 6 amino acids as shown in WO 97/08202 (AF-6), of said consensus sequence, or a modification thereof not altering the function of the polypeptide and/or peptide, such as by a peptide as shown in WO 97/08202 (AF-16), or in WO 97/08202 (AF-8).

Protein Antisecretory Factor (Protein AF) and peptides have previously been disclosed to normalize pathological fluid transport and/or inflammatory reactions, such as in the intestine and in the central nervous system after challenge with the cholera toxin (WO 97/08202). WO 97/08202 discloses structures of certain antisecretory proteins, and their active parts are characterized.

Food and feed with the capacity to either induce endogenous synthesis of AF or uptake of added AF have therefore been suggested to be useful for the treatment of oedema, diarrhoea, dehydration and inflammation in WO 97/08202. WO 98/21978 discloses the use of products having enzymatic activity for the production of a food that induces the formation of Protein Antisecretory Factor (Protein AF) after consumption. WO 00/038535 further discloses food products enriched and/or naturally rich in native Protein Antisecretory Factor (Protein AF) as such.

From the Swedish Patent SE 9000028-2 (publication No. 466,331) it is known that the formation of an antisecretory factor (AF) or an Protein Antisecretory Factor (Protein AF) (in SE 9000028-2 named ASP: also named FIL) can be stimulated by adding, to the animals' feed, certain sugars, amino acids and amides. The kinds and amounts of these substances to be used for the formation of an interesting amount of ASP is determined by a method disclosed in the patent. Briefly, this method involves measurement of a standardized secretion response in the small intestine of rat. From the patent it is evident that the induced ASPs formed direct the secretion of body fluid into the intestine. In said patent, the content or amount of natural antisecretory proteins is defined by its effect on the fluid secretion into the small intestine of laboratory rats having been challenged with cholera toxin (RTT-test). One ASP Unit (AF Unit/FIL Unit) corresponds to a 50% reduction of the fluid flow in the rat's intestine compared to a control without induced ASP. The antisecretory proteins are active in extremely small amounts and, therefore, it is often easier to determine them by their effect than by their mass.

From WO 98/21978 it is known that the formation of ASP can be induced in the body by consumption of a certain kind of food having enzymatic activity. The effect of the induction and, owing to that, the formation of ASP varies according to the individual and its symptoms and takes place with a strength and induction period unpredictable so far. However, they can be measured afterwards, and necessary corrections can be made with the guidance of said measurements. It is mentioned that the products may be malted cereals such as malted oats.

### Avenanthramides

Avenanthramides are a group of phenolic compounds comprising substituted N-cinnamoylanthranilic acids derived from cinnamic acid or a derivative thereof and anthranilic acid or a derivative thereof. The avenanthramides are mainly found in oat and have been reported to impart properties such as anti-inflammatory properties, antioxidant properties and anti-itch properties. In oat, the most abundant avenanthramides have been reported to be avenanthramides A, B, C, O, P and Q also called avenanthramides 2p, 2f, 2c, 2p_{d} and 2c_{d} as shown herein. The former nomenclature using capital letters is called Collin's nomenclature while the latter nomenclature is called Dimberg's modified nomenclature. In Dimberg's nomenclature the number refers to the anthranilic acid or a derivative thereof and the letter refers to the cinnamic acid or derivative thereof. For instance, “2” refers to 5-hydroxyanthranilic acid and “p” refers to p-coumaric acid. In addition, the letter “d” stands for double bond. In an example, avenanthramide A (2p) differs from avenanthramide O (2p_{d}) in the number of double bonds as shown in Scheme 1 below.

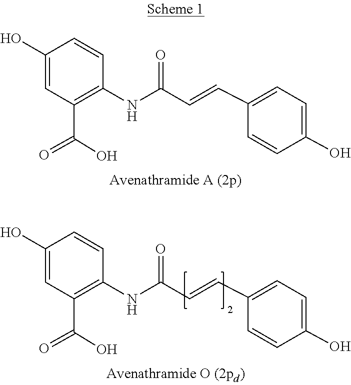

The report “A study of avenanthramides in oats for future applications” by Eléne Karlberg, Uppsala University School of Engineering, published in June 2010, discloses a method for enrichment of avenanthramides involving steeping and germination of oats at low pH. It is stated that an oat extract containing oat material subjected to this method would comprise positive physiological effects caused by avenanthramides and also beneficial effects originating from β-glucan.

WO 2010/108277 discloses methods for increasing the levels of avenanthramides in oats through false malting. Oats are first subject to induction or enhancement of a secondary dormancy, and then malted for up to 5 days at an elevated temperature. The malted but not germinated oats are then dried and used as is, or further processed or milled to produce food, feed, nutraceutical or personal care products and ingredients.

WO 2015/179676 discloses a composition and method for an avenanthramide-enriched, oat-based product having improved health effects. The oat-based product includes an avenanthramide ingredient having avenanthramides 2c:2p:2f in ratios comprising at least one of 1:1:1 or 1:2:2. The avenanthramide ingredient may be derived synthetically or recovered from processing raw oats into constituent oat fractions.

WO 2007/52153 states that it is known that the concentration of avenanthramides increase in the oat endosperm upon steeping in water. It is also stated that it has been reported that avenanthramides are thermally stable to steam processing, and that these studies may suggest that malting oats may contribute to increased antioxidant properties due to elevated levels of avenanthramides but that the role of malting to increase the antioxidant properties of oats has not been reported in the scientific literature.

It is an object of the present disclosure to provide a consumable product comprising malted oats and/or leachates of malted oats, which comprise substantially elevated levels of compounds, such as avenanthramides, which stimulate and/or induce endogenous production of Protein Antisecretory Factor (Protein AF), peptides and/or fragments thereof in a mammal after consumption and which, if consumed in an amount sufficient enough to induce said endogenous production of Protein Antisecretory Factor (Protein AF) and/or fragments thereof in said mammal, which increases cardiac output and promotes the recovery process of said mammal during and/or after physical activity.

In one embodiment of the present disclosure, such a consumable product for use in increasing cardiac output and promoting the recovery process of said mammal during and/or after physical activity comprises malted dehulled oats.

### SUMMARY OF THE INVENTION

It is a long-sought after need to increase cardiac output and to alleviate the negative consequences of strenuous exercises or competitions with dietary alterations. The present invention for the first time provides a means to ameliorate the negative effects of exercises on several critical parameters, such as but not limited to Hemoglobin (Hb), Erythrocyte Fluid Volume fraction (EFV) and creatine kinase (CK) and thus to simultaneously increase cardiac output and to decrease the recovery time necessary for horses and other mammals after physical activity, including the time for muscle recovery.

The present invention relates to the surprising insight that a consumable product comprising malted oats and/or a leachate of malted oats can, if consumed in an amount sufficient enough to induce endogenous production of Protein Antisecretory Factor (Protein AF) and/or fragments thereof in a mammal after consumption, promotes the level of oxygenation in blood and the recovery process of a mammal during and/or after physical activity. Said consumption of said consumable product before during and/or after said physical activity has for the first time been found to stabilizes the haemoglobin (Hb) value of said mammal after consumption of said consumable product at an average value between 133-139 g/L during and/or after physical activity, such as at an average value of no more than 139 g/L in a mammal during and/or after physical activity.

Further, a consumable product comprising malted oats and/or a leachate of malted oats for use according to the present invention further stabilizes the Erythrocyte Fluid Volume fraction (EFV) of a mammal during and/or after physical activity at a low value, such as of no more than 3% over resting value, such as at a value between 37-38% and/or stabilizes the creatine kinase value of said mammal at a low value of no more than approximately 1% over resting value.

In particular, a consumable product comprising malted oats and/or a leachate of malted oats for use according to the present invention has been found to be useful in increasing cardiac output and promoting the muscle recovery process of a mammal during and/or after physical activity.

The present invention thus in one embodiment relates to a consumable product comprising and/or consisting of malted dehulled oat and/or a leachate of said malted dehulled oat produced in accordance with a herein described malting process, which comprises malted dehulled oats and/or leachate of malted dehulled oats in an amount sufficient to increase the amount of antisecretory factor (AF) protein and/or fragments thereof in the subject's blood to at least about 0.7, such as at least 1 Units AF/mL blood, and to the use of the consumable product as food or feed and/or supplement to food or feed for humans and/or animals for use in increasing cardiac output and promoting the muscle recovery process of a mammal during and/or after physical activity.

Thus, a consumable product comprising malted oats and/or a leachate of malted oats for use according to the present invention increases level of oxygenation in the red blood cells, increases the mammal's stroke volume, ameliorates and/or prevents muscle damage caused by strains and/or tears, prevents paralysis during and/or after physical activity and/or decreases muscle recovery time after physical activity in a mammal if consumed in an amount sufficient enough to induce endogenous production of Protein Antisecretory Factor (Protein AF) and/or fragments thereof in a mammal after consumption.

In particular again, a consumable product comprising malted oats and/or a leachate of malted oats for use according to the present invention increases level of oxygenation in the red blood cells, increases the mammal's stroke volume and ameliorates and/or prevents muscle damage caused by strains and/or tears in an athletic mammal if consumed in an amount sufficient enough to induce endogenous production of Protein Antisecretory Factor (Protein AF) and/or fragments thereof in a mammal after consumption.

The present invention in conclusion relates to a consumable product comprising malted oats and/or a leachate of malted oats for a use as disclosed herein, wherein the consumption of the consumable product induces endogenous production of Protein Antisecretory Factor (Protein AF) and/or fragments thereof in a mammal, so that the mammal has at least about 0.5, such as at least 0.7, such as at least 1 Units AF/mL blood during and/or after physical activity. The induced higher level of Protein Antisecretory Factor (Protein AF) and/or fragments thereof in turn ameliorate the negative effects of exercises on several critical parameters, such as but not limited to Hemoglobin (Hb), Erythrocyte Fluid Volume fraction (EFV) and creatine kinase (CK) and thus simultaneously increase cardiac output and decrease the recovery time necessary for horses and other mammals after physical activity, including the time for muscle recovery.

As it stands to reason that other malted cereals with the ability to induce endogenous production of Protein Antisecretory Factor (Protein AF) and/or fragments thereof in a mammal after consumption will also be able to stabilize hemoglobin, creatine kinase and/or hematorcrit responses in a mammal during and/or after physical activity, the present invention in its widest scope relates to a consumable product comprising malted cereals and/or a leachate of malted cereals for a use as disclosed herein, wherein the consumption of the consumable product induces endogenous production of Protein Antisecretory Factor (Protein AF) and/or fragments thereof in a mammal, so that the mammal has at least 0.5 Units AF/mL blood during and/or after physical activity, such as so that the mammal has at least about 0.7, such as at least 1 Units AF/mL blood.

A consumable product comprising malted oats and/or a leachate of malted oats for use according to the present invention can be in a form selected from the group consisting of a food, feed, supplement to food or feed, a medicine and a nutraceutical for human and/or animal consumption.

In one embodiment, a consumable product for use according to the present invention comprises malted dehulled oats and/or a leachate of said malted dehulled oats, wherein said malted dehulled oats are produced by a malting process characterized by comprising the steps of:
a) dehulling oat kernels,
b) wet steeping of the dehulled oat kernels at a temperature from 5° C. to 20° C.,
c) germinating of said dehulled oat kernels at a temperature from 5° C. to 20° C.,
d) optionally repeating any one of steps b-c, and subsequent
e) drying of said dehulled oat kernels at no more than 80° C. air temperature, wherein the malted dehulled oats comprise i) avenanthramide D at a higher concentration as compared to the corresponding non-malted dehulled oats, such as at a concentration at least 100% higher than in the corresponding non-malted dehulled oats.

In one embodiment, said malted dehulled oats are produced by a malting process as described herein, wherein the wet steeping of the dehulled oat kernels in step b) is performed at a temperature from 7° C. to 15° C. for 1-5 days.

A consumable product for use according the present invention typically comprises malted dehulled oats and/or a leachate of said malted dehulled oats, wherein said malted dehulled oats are produced by a malting process as described herein, and wherein the malted dehulled oats further comprise one or more of:
(ii) avenanthramide A,
(iii) avenathramide C,
(iv) avenanthramide C methyl ester,
(v) (Z)-N-feruloyl 5-hydroxyanthranilic acid, and optionally
(vi) avenanthramide G, and

wherein the concentration of one or more of (ii), (iii), (iv), (v) and (vi) is higher as compared to in the corresponding non-malted dehulled oats.

A consumable product for use according to the present invention, produced by a process according to the present invention can in some embodiments further comprise a compound selected from the group consisting of guaiacol or a derivative thereof, L-tryptophan, DL-phenylalanine, and any combination thereof, wherein the concentration of at least one of said further compounds is higher as compared to the concentration of the same compound in the corresponding non-malted oat.

A consumable product comprising malted oats and/or a leachate of malted oats for use according to the present invention can be used for increasing cardiac output and/or promoting the recovery process of a mammal during and/or after physical activity which is predominantly aerobic exercise or predominantly anaerobic exercise.

A consumable product comprising malted oats and/or a leachate of malted oats for use according to the present invention can be used for increasing cardiac arrest and/or promoting the recovery process of a mammal during and/or after physical activity, wherein the mammal is a human being and/or an animal, or selected form the group consisting of equines such as horses and donkeys, dogs, and fur animals. In a presently preferred embodiment, the mammal is a horse.

A consumable product comprising malted oats and/or a leachate of malted oats according to the present invention can be used for preventing, ameliorating and/or treating hyperkalemia, such as hyperkalemic periodic paralysis (HYPP) in a horse.

A consumable product comprising malted oats and/or a leachate of malted oats for use according to the present invention can be in the form of a liquid, a solid or a combination thereof. Said consumable product is typically intended for daily human and/or animal consumption.

Said consumable product can be provided to the mammal at a dosage of at least 1 g/kg body weight/day, typically for at least 2 weeks.

A consumable product comprising malted oats and/or a leachate of malted oats for use according to the present invention can be a feed and/or feed supplement for livestock animals, such as a feed and/or feed supplement for equines.

The consumable product of the present invention typically regulates the fluid balance in the mammal's cells upon consumption. It further typically has anti-secretory properties, anti-diarrhoeal properties and/or anti-inflammatory properties.

The present invention consequently for the first time disclose a method for stabilizing the haemoglobin (Hb) value of a mammal after consumption of a consumable product at an average value between 133 -139 g/l, such as at no more than on average 139 g/l, during and/or after physical activity, comprising feeding a mammal a consumable product comprising malted oats and/or a leachate of malted oats for at least a period of 2 weeks before onset of the physical activity in an amount sufficient so that the mammal has at least 0.5 Units AF/mL blood at the onset of the physical activity.

The present invention further for the first time disclose a method for stabilizing the Erythrocyte Fluid Volume fraction (EFV) of a mammal during and/or after physical activity at a value of no more than 3% over resting value, and stabilizing the creatine kinase value of said mammal at a low value, such as of no more than 1% over resting value during and/or after physical activity, comprising feeding said mammal a consumable product comprising malted oats and/or a leachate of malted oats for at least a period of 2 weeks before onset of the physical activity in an amount sufficient so that the mammal has at least 0.5 Units AF/mL blood at the onset of the physical activity.

As well as a method for stabilizing the haematocrit volume fraction of a mammal at a value between 37-38%, such as at no more than 38% during and/or after physical activity, comprising feeding said mammal a consumable product comprising malted oats and/or a leachate of malted oats for at least a period of 2 weeks before onset of the physical activity in an amount sufficient so that the mammal has at least 0.5 Units AF/mL blood at the onset of the physical activity.

Another embodiment of the present invention is a method for increasing cardiac output, treating and/or preventing muscle damage caused by strains and/or tears in an athletic mammal, for preventing paralysis during and/or after physical activity and/or for decreasing muscle recovery time after physical activity in a mammal, comprising feeding said mammal a consumable product comprising malted oats and/or a leachate of malted oats for at least a period of 2 weeks before onset of the physical activity in an amount sufficient so that the mammal has at least 0.5 Units AF/mL blood at the onset of the physical activity.

A method according to the present invention comprises feeding a consumable product, wherein said consumable product comprises malted dehulled oats and/or a leachate of said malted dehulled oats, and wherein said malted dehulled oats are produced by a malting process disclosed herein.

A method according to the present invention comprises feeding a consumable product to a mammal which can be an animal, such as an equine, such as in particular a horse, and/or a human being.

A method according to the present invention comprises feeding a consumable product to a mammal, wherein said consumable product is provided to the mammal at a dosage of at least 1 g/kg/day. Typically, the mammal is fed the consumable product daily.

### Definitions and Abbreviations

In the present context, the term “performance” is defined as any form of work or forced physical activity. Work or physical activity can include walking, trotting, cantering, running, jumping, and turning. Therefore, a performance horses can include any horse that is actively ridden, trained or that may carry or pull a load. Since the performance activities of horses vary in both duration and intensity, feeding systems to address the nutrient requirements of these horses must also vary.

Proteins are biological macromolecules constituted by amino acid residues linked together by peptide bonds. Proteins, as linear polymers of amino acids, are also called polypeptides. Typically, proteins have 50-800 amino acid residues and hence have molecular weights in the range of from about 6,000 to about several hundred thousand Dalton or more. Small proteins are called peptides, polypeptides, or oligopeptides. The terms “protein”, “polypeptide”, “oligopeptide” and “peptide” may be used interchangeably in the present context. Peptides can have very few amino acid residues, such as between 2-50 amino acid residues (aa).

The term “antisecretory” refers in the present context to inhibiting or decreasing secretion and/or fluid transfer. Hence, the term “Protein Antisecretory Factor (Protein AF)” refers to a class of proteins capable of inhibiting or decreasing or otherwise modulating fluid transfer as well as secretion in a body.

In the present context, the terms an “antisecretory factor protein”, “Protein Antisecretory Factor (Protein AF)”, “AF-protein”, AF, or a homologue, derivative or fragment thereof, may be used interchangeably with the term “antisecretory factors” or “antisecretory factor proteins” as defined in WO 97/08202, and refer to an Protein Antisecretory Factor (Protein AF) or a peptide or a homologue, derivative and/or fragment thereof having antisecretory and/or equivalent functional and/or analogue activity, or to a modification thereof not altering the function of the polypeptide. Hence, it is to be understood that an “antisecretory factor”, “antisecretory factor protein”, “antisecretory peptide”, “antisecretory fragment”, or an “Protein Antisecretory Factor (Protein AF)” in the present context, also can refer to a derivative, homologue or fragment thereof. These terms may all be used interchangeably in the context of the present disclosure. Furthermore, in the present context, the term “antisecretory factor” may be abbreviated “AF”. Protein Antisecretory Factor (Protein AF) in the present context also refers to a protein with antisecretory properties as previously defined in WO 97/08202 and WO 00/38535. Antisecretory factors have also been disclosed e.g. in WO 05/030246.

The term “ASP” is in the present context used for “antisecretory protein” i.e. natural Protein Antisecretory Factor (Protein AF).

In the present context “AF activity” is measured as elevation of AF-Units in the blood after consumption of the consumable product of the present invention by inducing more than 0.5, such as at least 0.6, 0.7, 0.8, 0.9, 1, 1.5 or 2 Units AF/mL blood in a human or an animal. Increased AF activity is defined by its effect on the fluid secretion into the small intestine of laboratory rats having been challenged with cholera toxin (RTT-test/ligated loop assay). 1 ASP/Units AF/mL blood corresponds to (1 FIL-Unit) corresponds to a 50% reduction of the fluid flow in the rat's intestine compared to a control without ASP, i.e. corresponding approximately to 1.5 nmol AF protein per litre plasma (1.5 nmol/L).

AF activity can also be measured by the use of a kit, an assay and/or a method as described in WO 2015/181324 (Antisecretory Factor Complex Assay) for verifying effectiveness of a consumable product according to the present invention as compliance of human and/or animals to the same consumable product after consumption.

By “functional food product” is meant, in the present context, a food product having a salubrious function, i.e. having a beneficial effect on the health of man or an animal.

In the present context, the expression “pathologically high levels of body fluid discharge” means levels of body fluid discharge such as from intracellular fluid and/or extracellular fluid, the latter being selected from the group consisting of intravascular fluid, interstitial fluid, lymphatic fluid and transcellular fluid, that deviate from what is considered normal and/or healthy in a human and/or an animal. Specifically, the levels of body fluid discharge may be such that it may be considered by a health care professional such as a nurse or a physician appropriate to treat the patient. In the present context, the term “pathological” is used to in general describe an abnormal anatomical or physiological condition. The term “disease pathology” in general encompasses the causes, processes and changes in body organs and tissues that occur with human illness. Many of the most common pathological diseases are causes of death and disability.

Hyperkalemia in the present context interchangeable with “segregating hyperkalaemic periodic paralysis” (HYPP), or “Impressive Syndrome”. It is an autosomal dominant condition showing potassium-induced attacks of skeletal muscle paralysis. HYPP co-segregates with the equine adult skeletal muscle sodium channel a subunit gene, the same gene that causes human HYPP.

Hyperkalemia can be diagnosed as an excessive amount of potassium in the blood, which causes the muscles in the horse to contract more readily than normal. This makes the horse susceptible to sporadic episodes of muscle tremors or paralysis.

Symptoms of HYPP may include muscle twitching, unpredictable paralysis attacks which can lead to sudden death, and respiratory noises. Severity of attacks varies from unnoticeable to collapse or sudden death. The cause of death is usually respiratory failure and/or cardiac arrest.

AF: antisecretory factor, Protein Antisecretory Factor (Protein AF): Full-length AF protein (as shown in WO 97/08202, WO 07/126364)

AF-6: hexa peptide a fragment of Protein Antisecretory Factor (Protein AF) (as shown in WO 07/126364);

AF-16: a peptide composed of 16 amino acids a fragment of Protein Antisecretory Factor (Protein AF) (as shown in WO 97/08202, WO 07/126364);

AF-8: a septa peptide a fragment of Protein Antisecretory Factor (Protein AF) (as shown in WO 97/08202, WO 07/126364);

Octa peptide a fragment of Protein Antisecretory Factor (Protein AF) (as shown in WO 97/08202, WO 07/126364);

RTT: Method for measuring a standardized secretion response in rat small intestine, as published in SE 9000028-2 (publication number 466331) for measuring content of AF (ASP) in blood.

CK: creatine kinase (CK)

Hb: Hemoglobin (Hb)

cHb: Hb concentration in blood (cHb)

tHb: total Hb mass (tHb)

Hct: hematocrit (Hct)

EFV: Erythrocyte Fluid Volume fraction

tEFV: total red blood cell volume (Erythrocyte Fluid Volume fraction/tEFV) in circulation

g: gram(s)

mL: millilitre(s)

μL: microliter(s)

min.: minute(s)

vol: volume

UPLC: Ultra Performance Liquid Chromatography

V: Volt(s)

GHz: GigaHertz

LC-qTOF: Liquid Chromatography-quadrupole Time of Flight Mass Spectrometry (High Resolution Mass Spectroscopy)

RP: Reverse Phase

MS: Mass Spectroscopy

rpm: revolutions per minute

ppm: part per million

obiwarp-Ordered Bijective Interpolated Warping

mzML=mz(mass to charge ratio) ML

### BRIEF DESCRIPTION OF THE DRAWINGS

FIG. 1: Hemoglobin values g/L in horses fed a control diet or the same diet supplemented with SPC Performance in Stable 1 (a) and Stable 2 (b) in blood samples taken in Nov. before training (Hb 0), Day 1 after training (Hb1) and 3 days after training (Hb 3)

FIG. 2: Mean blood values taken Day 1 after training for μkat/L Creatine kinase (CK) and nmol/L Phosphorus (P) in horses in 2 stables.

FIG. 3: Mean values for changes in blood values (Hb and EFV) in horses fed either a Control diet or a diet supplemented with SPC Performance in 2 different stables.

FIG. 4: shows the chemical structure of avenanthramides A, B, C, D, G, 0, P and Q.

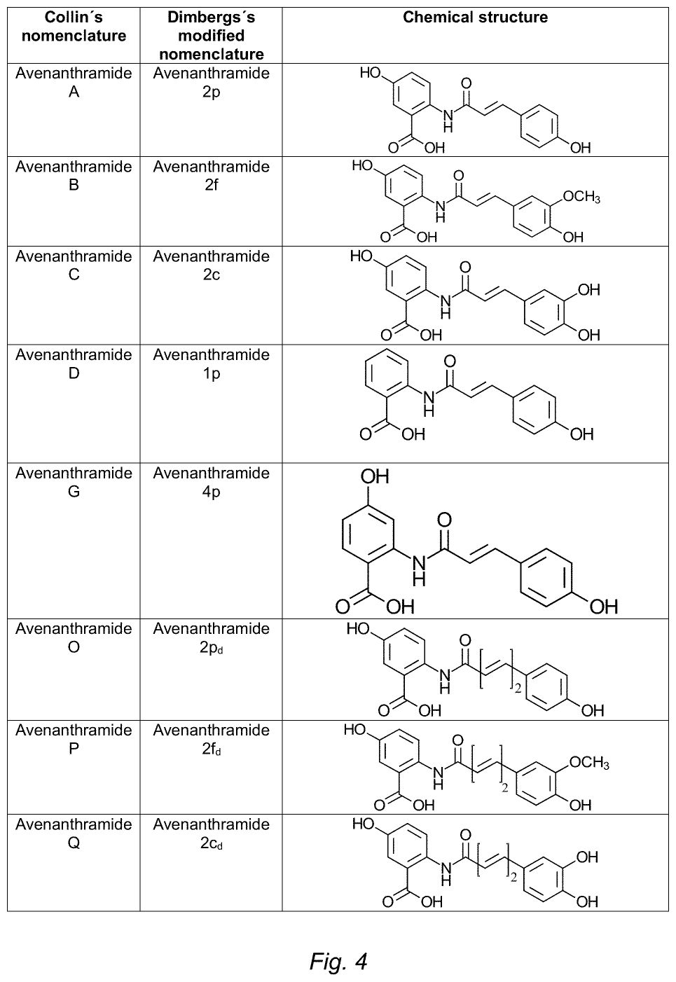

FIG. 5: shows the amount of avenanthramide 1p, i.e. avenathramide D, for the oat samples S1-S6.

### DETAILED DESCRIPTION

The present disclosure relates to means for promoting the recovery process of a mammal during and/or after physical activity, such as for treating and/or preventing muscle damage caused by strains and/or tears in a mammal, preventing paralysis during and/or after physical activity. In particular, the present invention relates to an effective means to decreasing muscle recovery time after physical activity in a mammal.

It is herein for the first time disclosed that a consumable product, comprising malted oats and/or a leachate of malted oats in an amount sufficient enough to induce endogenous production of Protein Antisecretory Factor (Protein AF) and/or fragments thereof (e.g. (as shown in WO 97/08202, WO 07/126364) in a mammal after consumption, stabilizes hemoglobin, creatine kinase and/or hematorcrit responses to low levels in a mammal during and/or after physical activity.

The malted oats and/or a leachate of malted oats comprised in the consumable product for use according to the present invention comprise in particular avenanthramide D at a concentration which is at least 100% higher as compared to in the corresponding non-malted oats and is obtained from a malting process comprising the steps of wet steeping and germinating oat at a temperature from about 5° C. to about 20° C., and subsequent drying said oat at no more than 80° C. air temperature.

### Effects of the Consumable Product on the Recovery Process

As can be seen in the experimental section, feeding a consumable product for use according to the present invention in the diet of horses, gave significantly lower values for the changes in both Hb and EFV compared to control animals after exercise.

There are significant effects of a consumable product for use according to the present invention on the absolute values for EFV compared to control and the lower values in EFV indicate a better recovery rate from hard training with a consumable product for use according to the present invention and further enhances the indications of the benefits of a consumable product for use according to the present invention in recovery.

In consequence, it is herein disclosed for the first time that a consumable product for use according to the present invention will limit changes in Hb and to limit EFV values to increases less than 3% compared to day 0 before training.

The lower creatine kinase values for a diet comprising a consumable product for use according to the present invention also indicate a better muscle status compared to the control diet.

The present invention relates to the surprising insight that a consumable product comprising malted oats and/or a leachate of malted oats can, if consumed in an amount sufficient enough to induce endogenous production of Protein Antisecretory Factor (Protein AF) and/or fragments thereof in a mammal after consumption, ameliorates several stressing effects of exercise, thereby increasing the cardiac output and promoting the recovery process of a mammal during and/or after physical activity. Consumption of a consumable product comprising malted oats and/or a leachate of malted oats as described herein has been found to stabilize the haemoglobin (Hb) value of a mammal after consumption of said consumable product, such as at an average value between 133-139 g/L during and/or after physical activity, such as at an average value of no more than 139 g/L in a mammal during and/or after physical activity.

Said consumption of said consumable product before during and/or after said physical activity has for the first time been found to stabilize the Erythrocyte Fluid Volume fraction (EFV) of a mammal during and/or after physical activity at a value of no more than 3% over resting value, such as at a value between 37-38%, and to stabilize the creatine kinase value of said mammal at a lower value than recorded in individuals which did not consume said consumable product.

Consumption of a consumable product according to present invention before during and/or after said physical activity has for the first time been found also to stabilize the creatine kinase value of said mammal at a value of approximately no more than 1% over resting value.

### Promoting Muscle Recovery Process—Decreasing Muscle Recovery Time after Physical Activity in a Mammal

In particular, a consumable product comprising malted oats and/or a leachate of malted oats for use according to the present invention has been found to be useful in promoting the muscle recovery process of a mammal during and/or after physical activity, thus effectively decreasing muscle recovery time after physical activity in a mammal.

A consumable product comprising malted oats and/or a leachate of malted oats for use according to the present invention has been found to be useful in ameliorating the negative effects of extreme and/or prolonged exercise on a number of physiological parameters which are prior well documented both in humans and horses, such as evaporative heat loss, fluid deficits, weight loss, uncorrected sweat loss, dehydration, electrolyte disturbances such as decrease in plasma concentrations of sodium, potassium, chloride, and calcium, and inadequate recovery of heart rate. During recovery from exercise, muscle protein synthesis increases in order to repair muscle tissue damaged during work.

As exemplified in the examples section, consumption of the consumable product of the present invention effects the blood values CK, Hb and EVF (Hematocrit) so that they do not rise as high during the physical exertion. In particle, the return to levels prior to exertion is quicker within the individuals having been feed the consumable product of the present invention, indicating quicker recovery rates and therefore a shorter rehabilitation including a decreased muscle recovery time after physical activity.

Thus, a consumable product comprising malted oats and/or a leachate of malted oats for use according to the present invention ameliorates and/or prevents muscle damage caused by strains and/or tears, prevents paralysis during and/or after physical activity and/or decreases muscle recovery time after physical activity in a mammal if consumed in an amount sufficient enough to induce endogenous production of Protein Antisecretory Factor (Protein AF) and/or fragments thereof in a mammal after consumption.

In particular again, a consumable product comprising malted oats and/or a leachate of malted oats for use according to the present invention ameliorates and/or prevents muscle damage caused by strains and/or tears in an athletic mammal if consumed in an amount sufficient enough to induce endogenous production of Protein Antisecretory Factor (Protein AF) and/or fragments thereof in a mammal after consumption.

### A Consumable Product

The present disclosure relates to a consumable product for use as described herein, comprising malted dehulled oats and/or a leachate of said malted dehulled oats comprising: (i) avenanthramide D, wherein the concentration of (i) is higher as compared to the corresponding non-malted dehulled oats, and wherein the consumable product induces endogenous production of Protein Antisecretory Factor (Protein AF) and/or fragments thereof in a subject after consumption.

SPC PERFORMANCE used in example 1 is horse feed and/or supplement comprising malted dehulled oats and/or a leachate of said malted dehulled oats comprising: (i) avenanthramide D, wherein the concentration of (i) is higher as compared to the corresponding non-malted dehulled oats, and wherein the consumable product induces endogenous production of Protein Antisecretory Factor (Protein AF) and/or fragments thereof in a subject after consumption. SPC PERFORMANCE used in example 1 is thus an exemplary consumable product according to the present invention.

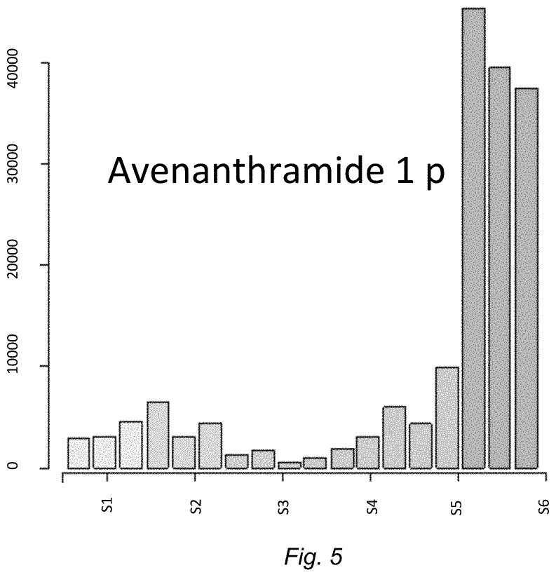

FIG. 5 shows the amount of avenanthramide 1p, i.e. avenathramide D, in a representative consumable product according to the present invention consisting of malted dehulled oats and/or a leachate of said malted dehulled oats.

The malted dehulled oats may further comprise one or more of:

(ii) avenanthramide A,

(iii) avenanthramide C,

(iv) avenanthramide C methyl ester,

(v) (z)-N-feruloyl 5-hydroxyanthranilic acid, and optionally

(vi) avenanthramide G,

wherein the concentration of one or more of (ii), (iii), (iv), (v) and (vi) is higher as compared to tin he corresponding non-malted dehulled oats.

The malted dehulled oats may also comprise:

(vii) a compound selected from the group consisting of guaiacol or a derivative thereof, L-tryptophan, DL-phenylalanine, and any combination thereof,

wherein the concentration of one or more of (vii) is higher as compared to in the corresponding non-malted dehulled oats. The guaiacol derivative may be ferulic acid, sinapic acid and/or p-coumaric acid.

A consumable product disclosed herein induces endogenous production of Protein Antisecretory Factor (Protein AF) and/or fragments thereof in a subject after consumption. The extent of the induction of said endogenous production of the Protein Antisecretory Factor (Protein AF) and/or fragments thereof may be adjusted by providing an appropriate amount of the consumable product to a subject in need thereof.

A consumable product comprising malted oats, produced with a malting process as described herein, comprises a combination of avenanthramide A, avenanthramide C methyl ester, avenanthramide D and certain compounds as described herein to such an increased amount that it induces endogenous production of Protein Antisecretory Factor (Protein AF) and/or fragments thereof in a subject after consumption.

Surprisingly, it was found that that the combination of the compounds above in the concentrations described herein increases the Antisecretory Factor (AF) activity, and/or improves the endogenous formation of AF in a subject after consumption.

Thus, there is provided a consumable product comprising malted oats and/or a leachate of said malted oats comprising in particular (i) avenanthramide D, wherein the concentration of (i) is higher as compared to the corresponding non-malted dehulled oats, and wherein the consumable product induces endogenous production of Protein Antisecretory Factor (Protein AF) and/or fragments thereof in a subject after consumption.

The malted oats and/or a leachate of said malted oats comprised in the consumable product may further comprise one or more of:

(ii) avenanthramide A,

(iii) avenanthramide C,

(iv) avenanthramide C methyl ester,

(v) (Z)-N-feruloyl 5-hydroxyanthranilic acid, and optionally

(vi) avenanthramide G;

wherein the concentration of one or more of (ii), (iii), (iv) (v) and (vi) is higher as compared to the corresponding non-malted oats.

The malted oats and/or a leachate of said malted oats comprised in the consumable product may further comprise:

(vii) a compound selected from the group consisting of guaiacol or a derivative thereof, L-tryptophan, DL-phenylalanine, and any combination thereof;

wherein the concentration of one or more of (vii) is higher as compared to the corresponding non-malted oats.

The guaiacol derivative described herein may be ferulic acid, sinapic acid and/or p-coumaric acid.

In one embodiment, a consumable product for use according to the present invention comprises malted oats and/or a leachate of said malted oats, wherein said malted oats are produced by a malting process characterized by comprising the steps of:
a) providing and optionally dehulling oat kernels,
b) wet steeping of the oat kernels at a temperature from 5° C. to 20° C.,
c) germinating of said oat kernels at a temperature from 5° C. to 20° C.,
d) optionally repeating any one of steps b-c, and subsequent
e) drying of said oat kernels at no more than 80° C. air temperature,

wherein the malted oats comprise i) avenanthramide D at a higher concentration as compared to the corresponding non-malted oats, such as at a concentration at least 100% higher than in the corresponding non-malted oats.

### Induction of Endogenous Production of Antisecretory Factor

The present invention in conclusion relates to a consumable product comprising malted oats and/or a leachate of malted oats for use as disclosed herein, wherein the consumption of the consumable product induces endogenous production of Protein Antisecretory Factor (Protein AF) and/or fragments thereof in a mammal, so that the mammal has at least 0.5 Units AF/mL blood during and/or after physical activity, such as so that the mammal has at least about 0.7, such as at least 1 Units AF/mL blood.

The consumable product described herein may comprise malted oats and/or a leachate thereof in an amount sufficient to induce endogenous production of Protein Antisecretory Factor (Protein AF) and/or fragments thereof in a subject after consumption. The specific amount of the consumable product may be adjusted depending on mammal to whom it is fed or is who is going to consume it. For instance, the consumable product may comprise malted oats and/or a leachate thereof in an amount sufficient to increase the amount of antisecretory protein and/or fragments thereof in the subject's blood to more than 0.5 Units AF/mL blood, such as to at least 0.6, 0.7, 0.8, 0.9 or at least 1 Units AF/mL blood. The skilled person may determine the amount using methods known in the art such as the RTT method (e.g. as disclosed in SE 9000028-2) and/or the Antisecretory Factor Complex Assay described in WO 2015/181324, or by any other well-known method, such as but not limited to by HPLC mass spectrometry, ELISA, western blotting, densitometric analysis, IP-MRM.

### Malting Process

The malting process described herein is a low-temperature malting process that allows malting of oats, in one embodiment dehulled oats, in a process that is easily scalable to industrial use.

In the process, the oat lot is refined by sieving and by using gravity tables so that the final 1000 grain weight exceeds 30 grams/1000 kernels. Such as that the final 1000 grain weight exceeds 30, 31, 32, 33, 34, 35, 36, 37, 38, 39, 40, 41, 42, 43, 44, 45, 46, 47, 48, 49, or 50 grams/1000 kernels.

The selected oat lot is in one embodiment dehulled by a dehuller. In the disclosed process, the dehuller is preferably a rotating disc with radial groves, but the person skilled in the art will understand that any commercially available dehuller can be used, as long as it leaves dehulled oats with the specified minimum germinability. A commercially available dehuller can be selected from the non-limiting group of Bühler BSSA Stratopact HKE50HP Ex and Streckel & Schrader. The feed and disc speed are typically selected so that 30-70% of the kernels are dehulled at each passage.

The germinability of the oat is tested to exceed 95%, such as no less than 80, 81, 82, 83, 84, 85, 85, 87, 88, 89, 90, 91, 92, 93, 94 or 95% in petri-dish, or at least 82%, such as at least 77, 76, 78, 79, 80, 81 or 82% in H₂O₂.

The selected oat kernels are steeped with cold water (w), optionally alternatingly in dried conditions (d) at temperatures between 5-15° C., or 7° C.-15° C., such as at temperatures not exceeding 5, 6, 7, 8, 9, 10, 11, 12, 13, 14 or 15° C., such as at a temperature between 5-12° C., 5-15, 12° C., 7-12° C., 12-15° C., 10-15° C. or 7-10° C., for a total of 1-3 days, such as for 20-26 hours, such as for 20, 21, 22, 23, 24, 25 or 26 hours, such as for no less than 1, 2 or 3 days. Kernel moisture content is herein kept between 30-50%, such as between 30-35%, 30-40%, 30-45%, 35-40%, 35-45%, 35-50%, 40-45%, 40-50% or 45-50%. The kernel moisture should in this process step not exceed 30, 35, 40, 45 or 50%.

In the present context, the malting comprises wet steeping in which the oat is partly or entirely soaked with water. Additionally, or alternatively, the wet steeping may involve spraying with water.

After steeping, the oat is germinated for 7-9 days at 5-20° C., preferably at 7-12° C., at 7-15° C., or at 12-15° C., such as for at least 7, 8 or 9 days at a temperature not exceeding 12, 13, 14, 15 or 20° C., such as at a temperature of 5, 6, 7, 8, 9, 10, 11, 12, 13, 14, 15 or 20° C.

The heat evolved is cooled by cold air. Due to the impermeable beds that can be formed, only shallow beds are used, with no more than 0.5 m bed height, such as with max 0.1, 0.2, 0.3, 0.4 or 0.5 m bed height. Any movement of the grains is performed at slow speed.

The germinated grain is initially dried at low air temperature not exceeding 35° C., such as at a temperature between 15-35, 20-35, 25-35 or 30-35° C. In the later stages of drying, when moisture content is below 20%, drying air temperature is raised to a maximum temperature of 65° C., max 65-70° C. or max 65-80° C. The drying air temperature should not exceed 80° C. at any time.

By this malting method, a healthy malted oat product with a high level of enzymatic activity is produced. In one embodiment, a healthy malted dehulled oat product with a high level of enzymatic activity is produced.

It has been found that the process for malting the oat impacts the properties of the consumable product into which it is incorporated. Importantly, the malting should take place at a low temperature such as from about 5° C. to about 20° C. and subsequent drying should take place at an air temperature of 80° C. or less. It will be appreciated that in this document the expression “a temperature of 80° C. or less” means a temperature equal to or less than 80° C.

Thus, there is provided a consumable product as described herein, wherein the malted oat is obtained from a process comprising the steps of: malting oat at a temperature from about 5° C. to about 20° C., and drying said oat at no more than 80° C.

In a further example, there is provided a consumable product as described herein, wherein the malted oat is obtained from a process comprising the steps of:

wet steeping of oat at a temperature from about 5° C. to about 20° C.,

germinating/growing at a temperature from about 5° C. to about 20° C.,

optionally repeating any one of steps a-b, and subsequent drying of said oat at no more than 80° C.

Steps a. and/or b. described herein may independently take place at a temperature of about 8° C. or from about 13° C. to about 15° C.

In one embodiment, said malted oats are produced by a malting process as described herein, wherein the wet steeping of the oat kernels in step b) is performed at a temperature from 7° C. to 15° C. for 1-5 days.

Further, the malting may comprise wet steeping in which the oat is partly or entirely soaked with water such as soaked with water for one hour or more. Additionally, or alternatively, the wet steeping may involve spraying with water.

The oat described herein may be a naked oat variety, it may be an oat variety with hull, and a de-hulled oat.

### Food, Feed, Supplement to Food or Feed

A consumable product comprising malted oats and/or a leachate of malted oats for use according to the present invention can be in a form selected from the group consisting of a food, feed, supplement to food or feed, a medicine and a nutraceutical for human and/or animal consumption.

The consumable product described herein may be food, feed, a food supplement, and/or a nutraceutical. The food or feed may be for human and/or animal consumption. Generally, food is intended for human consumption while feed is intended for animal consumption. The consumable product described herein may be a liquid, a solid and/or a combination thereof. For instance, the liquid may be a beverage. In a further example, the consumable product may be an infusion. When the food or feed is a solid it may be dry or semi-dry.

The food described herein may be a medical food. Additionally, or alternatively, the food described herein may be a FSMP, i.e. a food for special medical purposes. It will be appreciated that a FSMP may be food for individuals who suffer from certain diseases, disorders and/or medical conditions, and/or for subjects whose nutritional requirements cannot be met by normal foods. In a further example, the food described herein may be a nutraceutical. As used herein, a nutraceutical is a food or feed providing an extra health benefit in addition to basic nutritional value in food or feed. The food and/or food supplement for human consumption may be in the form of a liquid, a solid or a combination thereof. In an example, the food for human consumption may be in the form of a liquid, i.e. a liquid food for humans.

A consumable product comprising malted oats and/or a leachate of malted oats for use according to the present invention can be a feed and/or feed supplement for livestock animals, such as a feed and/or feed supplement for equines.

The feed described herein may be given to animals such as but not limited to athletic animals. The feed for animals may be in the form of a liquid, a solid or a combination thereof. In an example, the feed for animals may be in the form of a liquid, i.e. a liquid feed for animals.

In a particular example, the feed described herein is horse feed. In a further example, the feed described herein is dog feed.

In the present context, the term “feed” is used to describe materials of nutritional value fed to animals. Each species has a normal diet composed of feeds or feedstuffs which are appropriate to its kind of alimentary tract and which are economically sensible as well as being nutritious and palatable. Animals such as agricultural animals at pasture often have a diet which is very variable and subject to naturally occurring nutritional deficiencies. The feed disclosed herein may help to remedy or at least alleviate such deficiencies as well as disease, condition and/or symptom brought on by a stressful situation and or environment.

The presently disclosed feed can further comprise forage feed, such as hay, ensilage, green chop. i.e. any feed with a high cellulose content relative to other nutrients. The presently disclosed feed can further comprise feed grain such as cereal and other grains and pulses used as animal feed. The aforementioned feed grain may include wheat, barley, oats, rye, maize, peas, raps, rape seed, rape seed meal, soybean meal, and sorghum.

In a further example, the feed described herein may be provided in pelleted form.

The presently disclosed feed can further comprise feed supplements, i.e. nutritive materials which are feedstuffs in their own right, and which are added to a basic diet such as pasture and/or forage to supplement its deficiencies, such as minerals and aromatics. Feed supplements typically include trace elements and macrofeeds, feed additives or supplements, such as protein supplements and/or minor feed ingredients, such as essential amino acids and vitamins.

The consumable product can be a feed and/or food supplement in itself.

Albeit the present disclosure mainly is directed to the use of a consumable product in the form of food or feed, it is also envisaged that the consumable product may be administrated to a subject in other ways than oral intake. For instance, the consumable product may be provided in a form making it suitable for topical, ocular, subcutaneous and/or systemic administration.

The food described herein may form part of a functional food. For instance, the functional food may be muesli, bar, bread, biscuits, gruel, oatmeal, grains, flakes, pasta, omelette and/or pancake. In an example, the functional food is a beverage, or a food intended to drink. Alternatively, the functional food is not a beverage, or a food intended to drink but a solid or semi-solid foodstuff

Due to the presence of the malted oat and/or leachate of malted oat as described herein, the consumable product such as the food and/or feed possesses properties associated with induction of Protein Antisecretory Factor (Protein AF) and/or fragments thereof such as anti-secretory properties, anti-diarrhoeal properties and/or anti-inflammatory properties.

### Mammals

A consumable product comprising malted oats and/or a leachate of malted oats for use according to the present invention can be used for promoting the recovery process of a mammal during and/or after physical activity, wherein the mammal is a human being and/or an animal.

The mammal can be selected from recreational human beings, athletes, athletic animals and heavy-working humans and/or animals, such as employees or live-stock with labour-intensive workloads.

Typically, the mammal is selected form the group consisting of equines, horses, donkeys, and dogs. In a presently preferred embodiment, the mammal is a horse.

In one embodiment, the mammal is a race-horse. In one embodiment, the mammal is a greyhound. In one embodiment, the mammal is an endurance horse. In one embodiment, the mammal is a human long-distance runner. In one embodiment, the mammal is a long-distance running horse.

The feed described herein may be given to athletic animals or livestock animals.

### Exercise

A consumable product comprising malted oats and/or a leachate of malted oats for use according to the present invention can be used for promoting the recovery process of a mammal during and/or after physical activity which is predominantly aerobic exercise or predominantly anaerobic exercise.

Aerobic exercise is sometimes known as “cardio”—exercise that requires pumping of oxygenated blood by the heart to deliver oxygen to working muscles. Aerobic exercise stimulates the heart rate and breathing rate to increase in a way that can be sustained for the exercise session. In contrast, anaerobic (“without oxygen”) exercise is activity that causes you to be quickly out of breath, like sprinting or lifting a heavy weight. Predominantly aerobic exercises are in the current context selected from the non-limiting group including: Walking and trotting, some of the arena performance classes, i.e. reining, stadium jumping, and cutting, intersperse short bouts of anaerobic exercise with longer termed aerobic activities, western pleasure, horsemanship, and equitation, are largely aerobic, cardio machines, spinning, running, swimming, walking, hiking, aerobics classes, dancing, cross country skiing, and kickboxing. There are many other types.

Aerobic exercises can become anaerobic exercises if performed at a level of intensity that is too high.

Jumping a fence or pulling a heavy object are examples of a highly anaerobic type of exercise for horses. Quarter Horses and Thoroughbred races are predominately anaerobic. Predominantly, anaerobic exercises are in the current context selected from the non-limiting group including: Jumping, pulling, racing, heavy weight training, sprinting (running or cycling). Basically, any exercise that consists of short exertion, high-intensity movement is an anaerobic exercise.

### Medical Effects

Due to the presence of the malted oat and/or leachate of malted oat as described herein, the consumable product such as the food and/or feed possesses properties associated with induction of Protein Antisecretory Factor (Protein AF) and/or fragments thereof such as anti-secretory, anti-diarrheal properties and/or anti-inflammatory properties.

The consumable product described herein may be provided in the form of a medicament. Thus, there is provided a consumable product as described herein such as a functional food product and/or a pharmaceutical product for use as a medicament.

In one embodiment, a consumable product comprising malted oats and/or a leachate of malted oats according to the present invention can be used for preventing, ameliorating and/or treating hyperkalemia, such as hyperkalemic periodic paralysis (HYPP) in a horse.

A consumable product comprising malted oats and/or a leachate of malted oats for use according to the present invention can be in the form of a liquid, a solid or a combination thereof. Said consumable product is typically intended for daily human and/or animal consumption.

Said consumable product can be provided to the mammal at a dosage of at least 1 g/kg body weight/day, typically for at least 2 weeks.

The consumable product of the present invention typically regulates the fluid balance in the mammal's cells upon consumption. It further typically has anti-secretory properties, anti-diarrhoeal properties and/or anti-inflammatory properties.

In particular, the consumable product of the present invention regulates the hematocrit levels and RBC (red blood cells, erythrocytes). Especially the horse has a uniquely large reserve of red blood cells in the spleen which it utilizes during periods of hard work. This will obviously increase the hematocrit values during exercise. With dehydration and increases in RBC, the blood becomes thicker and harder to pump, putting an extra strain on the heart and other muscles. Therefore, it is in particular during extreme and/or long endurance exercise important to maintain an adequate fluid balance as well as adequate hematocrit values. This is true for other mammals as well, even if it is most studied in horses.

The consumable product of the present invention regulates the fluid balance in the mammal's cells upon consumption and regulates the hematocrit levels, thus effectively ameliorating the negative effects of athletic exercise, in particular on the heart and other muscles. As a consequence, consumption of the presently disclosed consumable product increases cardiac output of the mammal and increases the level of oxygen supply in the blood (oxygenation) during exercise and recovery.

As is e.g. documented in “Physiological Parameters of Endurance Horses Pre-Compared to Post-Race, Correlated with Performance: A Two Race Study from Scandinavia. Larson et al. 2013 ISRN Veterinary Science”. Changes in Hemoglobin (Hb), Erythrocyte Fluid Volume fraction (EFV) and creatine kinase (CK) will effect the recovery time for horses after physical activity. It is herein clearly documented that horses with lower levels of these values demonstrate a quicker recovery.

The consumable product of the present invention effectively ameliorates in particular the above-described negative changes in Hemoglobin (Hb), Erythrocyte Fluid Volume fraction (EFV) and creatine kinase (CK) and thus increases cardiac output and decreases the recovery time necessary for horses and other mammals, including humans, after physical activity, including the time for muscle recover, such as, but not limited to, the heart.

Thus, since the consumable product of the present invention ameliorates several of the negative effects of athletic exercise, the need for recovery time between exercises is reduced and/or decreased, including the time for muscle recovery.

A quicker recovery of muscles after exercise will in turn lead to preventing muscle damage caused by strains and/or tears and/or prevents paralysis during and/or after physical activity in a mammal, including but not limited to preventing, ameliorating and/or treating hyperkalemia, such as hyperkalemic periodic paralysis (HYPP) in a horse.

### Hemoglobin (Hb)

Hemoglobin (Hb) concentration is measured as g/L and indicates the capacity of blood to transport oxygen. Hb concentration changes due to elevation, but also due to exercise intensity. A standard resting value for a horse will vary between races and individual, values approximately between 120 och 160 g/L are considered standard resting values. Thus, in the present experiments, it could be seen that the Hb value did rise to an average value of between 133-139 g/L blood, which corresponds to a change of approximately 10%, such as between 10-15%, such as of no more than 10, 15 or 20%, such as at the most 20% from resting value.

Thus, a method is herein disclosed for stabilizing the haemoglobin (Hb) value of a mammal after consumption of a consumable product at an average value between 133-139 g/l, such as no more than 139 g/l, during and/or after physical activity, such as to a change of approximately 10%, such as between 10-15%, such as of no more than 10, 15 or 20%, such as at the most 20% from resting value, comprising feeding said mammal a consumable product comprising malted oats and/or a leachate of malted oats for at least a period of 2 weeks before onset of the physical activity in an amount sufficient so that the mammal has at least 0.5 Units AF/mL blood at the onset of the physical activity.

### Red Cell Volume Fraction (Hematocrit Value)

Hematocrit (Hct) Levels is the ratio of the volume of red cells to the volume of whole blood. Normal range for hematocrit is different between the sexes and is e.g. approximately 45% to 52% for human men and 37% to 48% for human women. Normal range for hematocrit is e.g. approximately 32% to 45% for horses.

Also provided is a method for stabilizing the haematocrit volume fraction of a mammal at a value between 37-38%, such as at no more than 38% during and/or after physical activity, comprising feeding said mammal a consumable product comprising malted oats and/or a leachate of malted oats for at least a period of 2 weeks before onset of the physical activity in an amount sufficient so that the mammal has at least 0.5 Units AF/mL blood at the onset of the physical activity.

### Erythrocyte Fluid Volume Fraction (EFV)

The present invention consequently for the first time also disclose a method for stabilizing the Erythrocyte Fluid Volume fraction (EFV) of a mammal during and/or after physical activity at a value of no more than 3% over resting value, comprising feeding said mammal a consumable product comprising malted oats and/or a leachate of malted oats for at least a period of 2 weeks before onset of the physical activity in an amount sufficient so that the mammal has at least 0.5 Units AF/mL blood at the onset of the physical activity.

Another embodiment of the present invention is a method for increasing cardiac output, treating and/or preventing muscle damage caused by strains and/or tears in an athletic mammal, preventing paralysis during and/or after physical activity and/or decreasing muscle recovery time after physical activity in a mammal, comprising feeding said mammal a consumable product comprising malted oats and/or a leachate of malted oats for at least a period of 2 weeks before onset of the physical activity in an amount sufficient so that the mammal has at least 0.5 Units AF/mL blood at the onset of the physical activity.

A method according to the present invention comprises feeding a consumable product, wherein said consumable product comprises malted dehulled oats and/or a leachate of said malted dehulled oats, and wherein said malted dehulled oats are produced by a malting process disclosed herein.

A method according to the present invention comprises feeding a consumable product to a mammal which can be an animal, such as an equine, such as in particular a horse, and/or a human being.

A method according to the present invention comprises feeding a consumable product to a mammal, wherein said consumable product is provided to the mammal at a dosage of at least 1 g/kg/day. Typically, the mammal is fed the consumable product daily.

The present disclosure will be further explained hereinafter by means of non-limiting examples and with reference to the appended drawings.

### REFERENCES

1. Lange and Lönnroth, 2001
2. WO 97/08202;
3. WO 05/030246;
4. WO 2007/126364;
5. WO 2018/015379
6. WO 98/21978
7. WO 07/126363
8. SE 9000028-2 (publication No. 466,331)
9. A study of avenanthramides in oats for future applications” by Eléne Karlberg, Uppsala University School of Engineering, published in June 2010
10. WO 2010/108277
11. WO 2015/179676
12. WO 2007/52153
13. U.S. Pat. No. 4,581,847
14. WO 2007/117815
15. WO 2017/09004
16. WO 00/38535
17. WO 201 5/1 81 324
18. Food Chemistry 253 (2018) 93-100
19. Physiological Parameters of Endurance Horses Pre-Compared to Post-Race, Correlated with Performance: A Two Race Study from Scandinavia. Larson et al. 2013 ISRN Veterinary Science

### EXAMPLES

### Example 1

The effects of SPC PERFORMANCE (a consumable product according to the present invention) on recovery after exercise assessed by comparison of blood values for Hemoglobin, Hematocrit, Creatine kinase, Phosphorous, Protein, and Creatinine.

### Methods

The effects of supplementing horse feed with SPC PERFORMANCE were examined in two (2) racing stables with Standardbred horses in training. In both stables, horses were divided randomly between two groups. In Stable 1 there were 12 horses in Control group and 11 in SPC PERFORMANCE group. In Stable 2 there were 9 horses in Control and 10 horses in SPC PERFORMANCE group.

Horses were fed daily SPC PERFORMANCE with start in July and continued for at least 3 months. SPC PERFORMANCE was fed at a dose of 500 g per day and horse and administered in 1 kg of a pelleted feed (Krafft Max Balance). Feeding strategies differed between stables but SPC PERFORMANCE feeding was the same.

Blood samples were drawn on day 0 before training and then again days 1 and 3 after training. Samples were taken in July from all horses before the start of SPC PERFORMANCE feeding and then again in Nov. Samples were analyzed by a commercial laboratory.

The training schedule and set-up was the same in July and Nov.

Samples were analyzed for a range of parameters normal in assessing the condition of the horses. Hematocrit and hemoglobin were analyzed for all days but Creatine kinase, Phosphorous, Protein, and Creatinine were analyzed only in samples from day 1 after training.

Blood values for hematocrit and hemoglobin were analyzed using One-Way Anova for comparison of changes in absolute values before and after training both in July and in November.

In addition, results from the 2 stables were combined into one (1) data set and changes in absolute values for blood parameters were recalculated to changes expressed as percentages (%). Comparisons of changes between Control horses and SPC PERFORMANCE fed horses were analyzed using the GLM procedure in Minitab 16® with diet, stable and day. Not all parameters had a normal distribution and where necessary values were transformed.

### Results

### Hemoglobin and Hematocrit in Stables 1 and 2

In July before the start of feeding SPC PERFORMANCE all horses exhibited the same pattern of increases for Hemoglobin and Hematocrit after training and the increase after training was significant. There were no differences between groups on Day 0, Day 1 or Day 3 before the start of feeding SPC PERFORMANCE. There were differences between the 2 stables in absolute values.

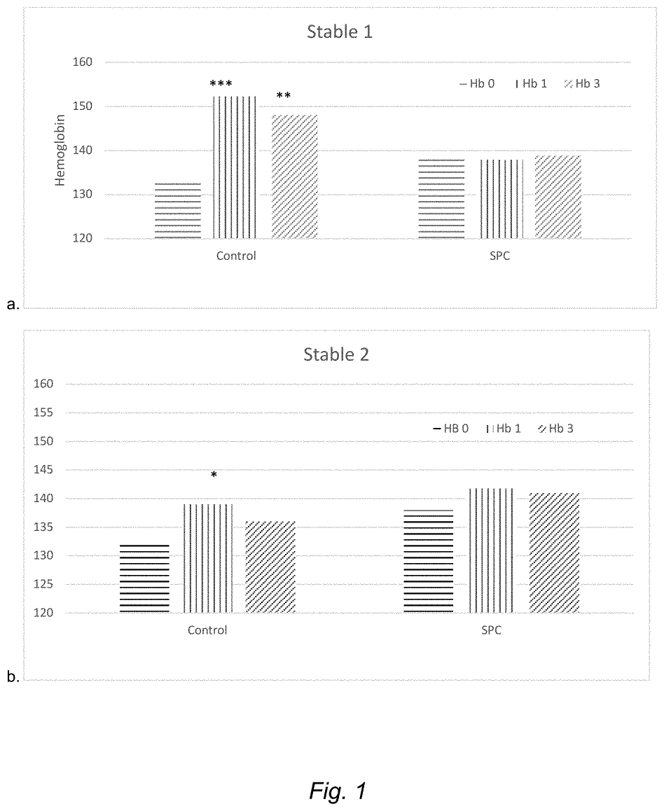

In November after 3 months of SPC PERFORMANCE consumption the increase in Hemoglobin and Hematocrit (results for EFV not shown) after training was, for the Control group, similar to the pattern in July and the increase was statistically significant (see Hb values for stable 1 in FIG. 1a). For the SPC PERFORMANCE group there were no significant increases in Hemoglobin and Hematocrit (results for EFV not shown) on days 1 or 3 after training. This was seen in both stables.

In Stable 2 hemoglobin values were lower in the control group but the increase in hemoglobin after training was significant whereas there was no significant increase in the SPC PERFORMANCE group (FIG. 1b). The lower significance compared to Stable 1 was due to more variation in the results from Stable 2.

While results from both stables indicate the same positive effect of SPC PERFORMANCE on recovery the variation makes it difficult to quantify the magnitude of the changes. Therefore the changes were analyzed as differences from the values at Day 0 before training in Nov.

### Combined Results from Stables 1 and 2

To express the changes in relative values results from the 2 stables were combined in one data set and the effect of SPC PERFORMANCE was analyzed using a General Linear Model (GLM).

In the GLM the factors diet (Control and SPC PERFORMANCE), Days (Day 1 and 3) and stable (1 and 2) are included as wells as the interaction between diet and day.

For blood parameters other than Hemoglobin (Hb) and Hematocrit (EFV) samples were only from Day 1 after training and changes from day 0 before training could not be analyzed. For these blood parameters the comparison presented is between diets (Control and SPC PERFORMANCE) and Stables.

Values for several parameters were not normally distributed and could not be analyzed Creatinine Kinase did not have a normal distribution but could be analyzed after transformation to log values (Ln).

Due to large variation between horses and stables the models did not have a very high accuracy.

The effect of stable was significant in the model.

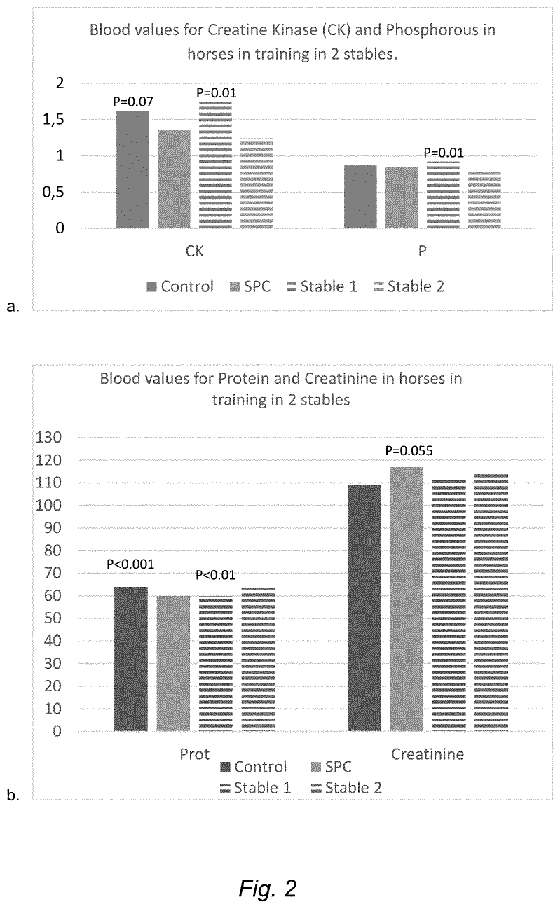

FIG. 2a shows mean blood values for Creatine kinase (CK) and Phosphorus levels in horses in 2 stables.

Higher levels of CK is a possible indication of muscle damage and the lower values for the SPC PERFORMANCE group is positive (FIG. 2a). That diet differences were not highly significant (P=0.07) could be due to the large variation between stables. The higher levels for the Control, group are noteworthy as CK usually peaks 6-12 t after an injury while the values are in samples taken 24 h after training.

Lower Phosphorus levels are normal after training. There were no differences due to diets (FIG. 2a) but the stables differed significantly, possibly due to differences in training schemes.

Protein values refer to total protein (g/L). Higher values for the Control diet can indicate a lower fluid volume in the blood (FIG. 2b). Creatinine (μmol/L) is normally excreted in the urine and higher levels can indicate conditions affecting the kidneys, such as dehydration. Despite differences between diets and stables all values were within normal ranges and did not indicate abnormal physiological states.

Even though several parameters such as AST, GT and Mg could not be analyzed statistically due to a non-normal distribution the values were within normal ranges and did not indicate any problems with the liver or the kidneys in either the Control or the SPC PERFORMANCE group.

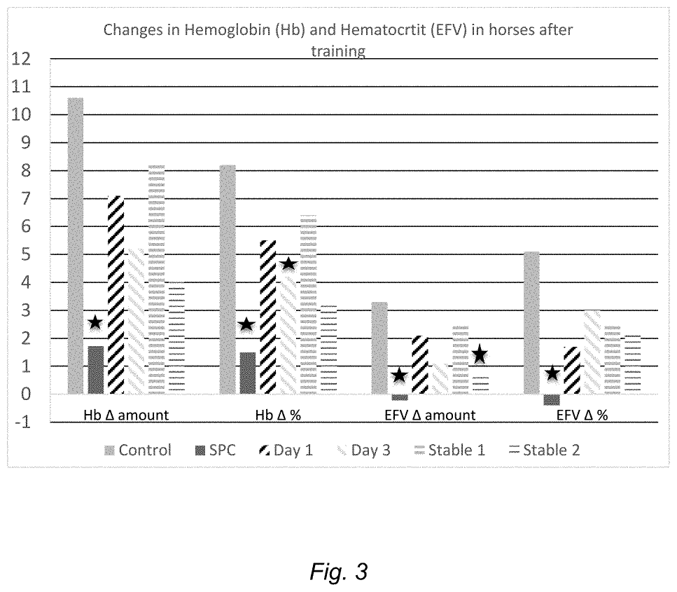

FIG. 3 shows mean values for changes in blood values (Hb and EFV) calculated as differences between values on Day 0 and Days 1 as well as Day 3 in horses fed either a Control diet or a diet supplemented with SPC PERFORMANCE in 2 different stables.

Although there a high variation of Hb values was noted between the stables, significant differences for the absolute hemoglobin values (P=0.0002) were noted in the combined data set between, days 1 and 3 when stables were analyzed individually. The absolute values for hematocrit (EFV) differed between diets and stables, with lower values for SPC PERFORMANCE and Stable 2.

In the combined data set the increase in Hb values did not exceed more than 2 g/L or 2% compared to the Hb at Day 0 for the SPC PERFORMANCE group (FIG. 3). This was significantly different from the values for the Control group which exceeded more 10 g/L and 8% compared to Day 0.

In the combined data set EFV decreased for the SPC PERFORMANCE group which was significantly different from the Control group in which EFV increased with more than 3 percentage units or more than a 5% increase compared to Day 0 (FIG. 3).

There were significant differences between Days 1 and 3 for Hb values as could be expected.

There were significant differences between stables for changes in absolute values for EFV.

### Conclusions

Feeding SPC PERFORMANCE in the diet gave significantly lower values for the changes in both Hb and EFV compared to control which indicates that SPC PERFORMANCE can aid in recovery.

There were significant effects of SPC PERFORMANCE on the absolute values for EFV compared to Control and the lower values in EFV indicate a better recovery rate from hard training with SPC PERFORMANCE and further enhances the indications of the benefits of SPC PERFORMANCE in recovery.

SPC PERFORMANCE limits changes in Hb to increase no more than 10-20% and EFV to increases less than 3% compared to Day 0 before training.

The lower Creatine kinase values for the SPC PERFORMANCE diet indicate a better muscle status compared to the Control diet but there is no information concerning changes over time.

1.-43. (canceled)

44. A method of increasing cardiac output, the level of oxygen supply in the blood, and/or promoting muscle recovery during and/or after physical activity in a mammal, comprising
providing the mammal with a consumable product comprising malted dehulled oats and/or a leachate of malted dehulled oats in an amount sufficient to induce endogenous production of Protein Antisecretory Factor (Protein AF) and/or fragments thereof in a mammal after consumption of the product, wherein the consumable product comprises avenanthramide D at an at least 100% higher concentration compared to a product which comprises non-malted dehulled oats but is otherwise identical to the consumable product.

45. The method of claim 1, wherein the consumption of the consumable product by the mammal stabilizes the average haemoglobin (Hb) value of the mammal at a value between 133-139 g/L during and/or after physical activity.

46. The method of claim 1, wherein the consumption of the consumable product by the mammal stabilizes the Erythrocyte Fluid Volume fraction (EFV) of the mammal during and/or after physical activity at a value of no more than 3% over resting value.

47. The method of claim 1, wherein the consumption of the consumable product by the mammal stabilizes the creatine kinase value of the mammal at a low value.

48. The method of claim 1, wherein the average haemoglobin value of the mammal after consumption of said consumable product is stabilized at an increased value of no more than approximately 10-20% during and/or after physical activity.

49. The method of claim 1, wherein the consumption of the consumable product by the mammal stabilizes the Erythrocyte Fluid Volume fraction (EFV) of the mammal during and/or after physical activity at a value between 37-38%.

50. The method of claim 1, wherein the consumption of the consumable product by the mammal stabilizes the creatine kinase value of a mammal at a low value of no more than 1% over resting value.

51. The method of claim 1, wherein the consumption of the consumable product by the mammal decreases muscle recovery time after physical activity in the mammal, ameliorates and/or prevents muscle damage caused by strains and/or tears and/or prevents paralysis during and/or after physical activity in the mammal.

52. The method of claim 1, wherein the consumption of the consumable product by the mammal induces endogenous production of Protein Antisecretory Factor (Protein AF) and/or fragments thereof in the mammal, so that the mammal has at least 0.5 Units AF/mL blood during and/or after physical activity.

53. The method of claim 1, wherein the consumable product is a food or feed and/or supplement to food or feed for human and/or animal consumption.

54. The method of claim 1, wherein the consumable product is a medicine and/or a nutraceutical.

55. A process for preparing a consumable product comprising the steps of:
a. dehulling oat kernels,
b. wet steeping of the dehulled oat kernels at a temperature from 5° C. to 20° C.,
c. germinating of the dehulled oat kernels at a temperature from 5° C. to 20° C.,
d. optionally repeating any one of steps b-c, and subsequently
e. drying the dehulled oat kernels at no more than 80° C. air temperature.

56. The process of claim 55, wherein the wet steeping of the dehulled oat kernels in step b. is performed at a temperature from 7° C. to 15° C. for 1-5 days.

57. The method of claim 1, wherein the mammal is a human being and/or an animal.

58. The method of claim 57, wherein the animal is selected from the group consisting of equines and dogs.
